(19) United States
(12) Patent Application Publication
(10) Pub. No.: US 2004/0175771 A1
(43) Pub. Date: Sep. 9, 2004

(54) At-home cancer test

(76) Inventor: John Angelo Swetledge, Walker, LA (US)

Correspondence Address: John Angelo Swetledge, 30735 N. Walker Rd., Walker, LA, 70785, US

(21) Appl. No.: 10/376,673

(22) Filed: Mar. 4, 2003

(51) Int. Cl. G01N 33/574

(52) U.S. Cl. 435/7.23

(57) ABSTRACT

This utility patent changes the chemical/biological formula of a home pregnancy test by narrowing the antigen from hCG/CGH to the beta-subunit only of hCG/CGH since the alpha-subunit of hCG/CGH is entirely too similar to that of the alpha-subunits of other hormones found to be naturally occurring in the human body. These hormones include LH, FSH and TSH.

This utility patent also asserts that there are a variety of monoclonal antibodies at the disposal of the industry with which a manufacturer can effectively and adequately build and market an in-vitro urine assay that will detect even trace amounts of the antigen beta-hCG/CGH, which we claim to be the definitive cancer bio-marker.

### BACKGROUND OF THE INVENTION

The At-Home Cancer Test mimics the function of laboratory tests administered by medical professionals at hospitals and clinics.

The test itself is similar in form to that of a home-pregnancy test, and it will include the following: a packaging box containing a urine specimen container shrink-wrapped separate from the fluorescent marker and monoclonal antibody impregnated test strips as well as illustrated instructions.

My test will detect the beta-subunit of hCG/CGH, if present, in the test takers urine.

The At-Home Cancer Test will detect any and all perceived forms of carcinoma earlier in many cases than conventional laboratory tests because the yield of the antigen, beta-hCG/CGH, is higher in urine than in blood.

My invention parallels the in-vitro laboratory test designed by the late Dr. Manuel Navarro, Professor of Medicine and Surgery, University of Santo Tomas in Manila, Philippines.

Although the processes are alike, my test is not quantitative, but rather, qualitative.

This means the result is expressed by a simple present or not present.

The At-Home Cancer Test will also rule out potential interference and false positives due to the presence of similar hormones such as: LH, FSH, or TSH by distinguishing the beta-subunit of hCG/CGH which differs greatly from that of the other above listed hormones.

The At-Home Cancer Test relates in general, to in-vitro assays and their laboratory/clinical counterparts.

As there is no all-purpose/general carcinoma assay for at home use on the market today, it will be necessary to contrast my test to that of clinical serum assays as well as the tissue specific, at-home, in-vitro assays currently on the market.

1. At-home, in-vitro assays cited:

a. A bladder specific cancer test has been submitted to the FDA for 510 (k) approval in June 2002 by Matritech. This test is called BladderChek. This information is taken from a report in the Jul. 10, 2002 Biotech Week.

2. Clinical, in-vitro assays cited:

a. A prostate specific cancer test designed and created by: Jennifer L. Sanders, Naibedya Chattopadhyay, Olga Kifor, Toru Yamaguchi and Edward M. Brown, not yet submitted to the FDA for 510 (k) approval, has been made public by a report in the December, 2001 The American Journal of Physiology.

It is broadly accepted, within the Oncology community, that hCG/CGH is the definitive biomarker for Chorio-Carcinoma and Testicular tumors. It is however the beta-subunit of hCG/CGH that, as an antigen, is distinguishable from any other hormone or hormonal sub-unit found within the human body.

Since hCG/CGH is found in every perceived form of cancer and no carcinoma has ever demonstrated the absence of this hormone, and the beta-subunit, rather than the alpha-subunit of hCG/CGH is unique and entirely different from any other hormone or hormonal sub-unit found within the human body, a great deal of research and study has been put forth by many nations and languages to determine if this is truly the definitive cancer bio-marker.

When Dr. J. L. Vaitukaitis developed a monoclonal anti-body against the beta-hCG antigen in 1972, the studies were wide open for Dr. Manuel Navarro, University of Manila, Philippines, to develop a general and quantitative cancer test for use in the clinics and laboratories.

I have removed the quantitative aspects of the test introduced by Dr. Navarro and repackaged the components into an easy-to-use, over-the-counter, qualitative, in-vitro assay for public use.

### BRIEF SUMMARY OF THE INVENTION

Unique Features and Benefits of my invention:

In-home use

Administered by self or someone trusted

Personal discretion

Highly accurate

Inexpensive relative to lab tests

Convenient to users schedule

Early detection of all perceived forms of carcinomas

Caters to individuals with doctor or hospital/clinical phobias

Painless as opposed to conventional clinical tests

Faster, near instant results

As previously noted, At-Home Cancer Tests on the market today are tissue specific and will not indicate the presence of carcinoma in any part of the body other than that which is being tested.

Since cancers can be found in all parts of the human body, albeit with a greater frequency in some locations over others, and there remains but a few very specialized tissue assays for public access, a great number of malignancies go undetected until a symptom or some discomfort manifests itself and persuades the sufferer to seek medical attention.

For a large number of individuals, the very narrow window of opportunity for early detection has passed, along with greater opportunities for successful treatment.

Being a general/non specific cancer test with an effectiveness equal to that of clinical serum tests, this particular cancer test can fill a great void on our pharmaceutical shelves and encourage those with affirmative results from the examination to seek medical attention for locational diagnostics and targeted treatments. The early detection provided by this test increases ones chances of survival by allowing the treatment administering physician ample time to develop an appropriate course reaction.

Because a great number of individuals do not hold medical insurance and cannot afford the rising cost of routine physician administered medical examinations, an inexpensive substitute is in order.

The At-Home Cancer Test is just that, inexpensive and can be as routine as desired.

An easy to use at home cancer test can also address issues some people have with clinical visits. An individual or someone trusted can administer this test in the privacy of their own home with only themselves or whomever they choose being privileged to the results of the examination.

### DESCRIPTION OF DRAWINGS

| A - protective cap | A - decorative cap |
|---|---|
| B - absorbent tip | B - absorbent tip |
| C - test result window | C - splash guard |
| D - control window | D - finger grip |
| E - contoured thumb grip/holding area | E - square window |
| F - sealed splash guard | F - round window |
| A - result window | A - exposed absorbent pad |
| B - thumb grip | B - immersion level indicator |
| | mark |
| C - collection window | C - positive mark |
| | D - control mark |
| | E - thumb/finger grip |
| A - test strip material | A - specimen cup |
| B - immersion level indicator mark | B - minimum fill line |
| C - proof result pad | C - volume requirement label |
| D - alternative result pad | |

### ADDITIONAL NOTES

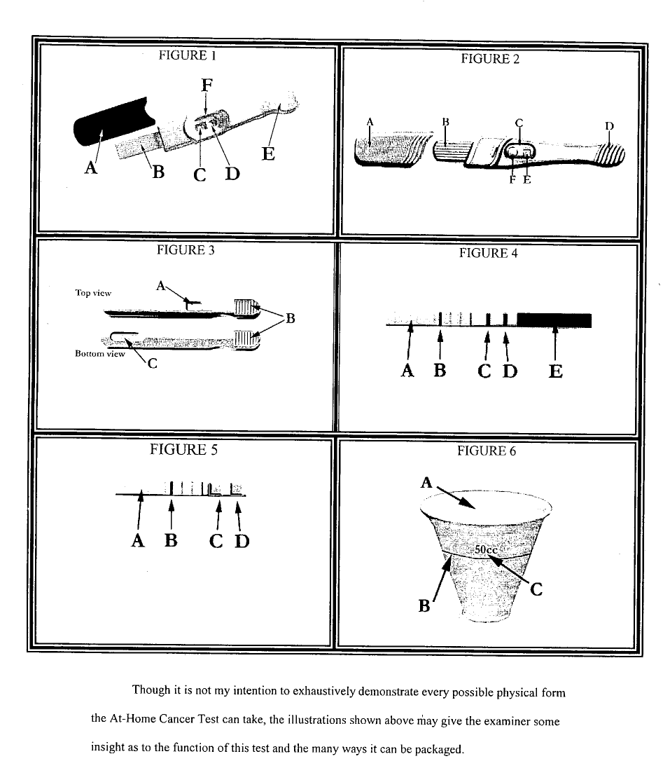

Absorbent pads can be natural fiber, paper or synthetic fiber. FIG. 1 is of a practical design. FIG. 2 is of a decorative design. FIG. 3 is of a fully encapsulated design with a specimen collection window instead of an exposed absorbent tip. FIG. 4 is of a simple design. FIG. 5 is of an inexpensive strip design. The strip materiel in FIG. 5 can be of a natural fiber, plastic, paper, synthetic fiber or any materiel found not to be in interference with the antibody/antigen reaction. FIG. 6 is of a specimen container cup.

### DETAILED DESCRIPTION OF THE INVENTION

The process by which this test is manufactured is near to that of the home pregnancy test kits currently available for public use with the only exception being that of which antigen is being tested for.

The distinguishing difference is this: the applied antibody must be monoclonal and only react to the beta-subunit of the hCG/CGH molecule.

Any suitable monoclonal antibody for reaction against the antigen, beta-hCG/CGH, is the primary and only difference between home pregnancy test kits and the At-Home Cancer Test.

All in-vitro, home pregnancy test kits currently being manufactured use anti-bodies against the whole hCG molecule. This process of using polyclonal antibodies includes the alpha-as subunit as part of the antigen.

For carcinogenic trophoblastic cells to be distinguished from that of pregnancy trophoblastic cells, only the beta-subunit of human Chorionic Gonadotropins can be used as the antigen.

Any monoclonal antibody that reacts sufficient in a manner do distinguish itself against the beta-hCG/CGH antigen will suffice as a key property of the At-Home Cancer Test.

### Definition of Terms

hCG—<hormone> <tumor marker> HUMAN CHORIONIC GONADOTROPIN A hormone that is produced by the developing placenta and by the fertilized egg after implantation in the uterine wall. This hormone is measured in the blood to determine pregnancy. hCG increases in quantity through the first trimester of pregnancy and begins to taper off after 85 days. Serum human chorionic gonadotrophin testing is accurate 48 hour post conception. Urine hCG testing is a popular method of home pregnancy determination as human chorionic gonadotrophin can be detected in urine.

CGH—<hormone> CHORIONIC GONADOTROPIC HORMONE same as above

LH—<hormone> LUTEINIZING HORMONE A small glycoprotein hormone secreted by the anterior pituitary gland that plays an important role in controlling ovulation and in controlling secretion of hormones by the ovaries and testes.

FSH—<hormone> FOLLICLE-STIMULATING HORMONE An acidic glycoprotein secreted by the anterior pituitary gland. In women, follicle-stimulating hormone stimulates the development of ovarian follicles (eggs) and stimulates the release of estrogen. In men, follicle-stimulating hormone stimulates the production of sperm.

TSH—<hormone> THYROID STIMULATING HORMONE <endocrinology> A polypeptide hormone (28 kD), secreted by the anterior pituitary gland, that activates cyclic AMP production in thyroid cells leading to production and release of the thyroid hormones (T4 and T3).

IN-VITRO—<within a glass, observable in a test tube, in an artificial environment> a physical examination of a human tissue or fluid sample performed external to the human body.

ASSAY—<procedure> determination of the amount of or the presence of a particular constituent of a mixture.

BIOMARKER—<biochemistry> A specific biological chemical in the body which has a particular molecular feature that makes it useful for measuring the progress or presence of disease or the effects of treatment. A specific bodily produced indicator of the presence of disease or foreign agent, which can serve as an antigen.

FLUORESCENT MARKER—an antibody or chemical belonging to an application designed to produce a change in color with exposure to an assigned antigen/antibody reaction.

IMPREGNATED—filled or saturated with a substance, mixture or formula of substances. Having a substance, mixture or formula of substances applied to a surface for direct exposure.

ANTIBODY—An immunoglobulin molecule that has a specific amino acid sequence by virtue of which it interacts only with the antigen that induced its synthesis in cells of the lymphoid series (especially plasma cells) or with antigen closely related to it. Antibodies are classified according to their ode of action as agglutinins, bacteriolysins, haemolysins, opsonins, precipitins, etc.

MONOCLONAL ANTIBODY—an antibody that is mass-produced in the laboratory from a single clone and that recognizes only one antigen.

POLYCLONAL ANTIBODY—Derived from different types of cells. An antibody produced by several clones of B lymphocytes as would be the case in a whole animal. Usually refers to antibodies raised in immunized animals, whereas a monoclonal antibody is the product of a single clone of B lymphocytes, usually maintained in vitro.

QUANTITATIVE—Denoting or expressible as quantity, relating to the proportionate quantities or to the amount of the constituents of a compound. Expressing a measurement of, or degree of, the presence of a substance.

QUALITATIVE—concerned only with the presence of a substance. Determination of the nature, as opposed to the quantity, of each of the elements composing a substance.

SERUM—<haematology> of, or pertaining to, drawn blood. Blood serum, the clear liquid that separates from blood on clotting.

ANTIGEN—Substance, which is capable, under appropriate conditions, of inducing a specific immune response and of reacting with the products of that response, that is, with specific antibodies or specifically sensitized T-lymphocytes, or both. Antigens may be soluble substances, such as toxins and foreign proteins, or particulates, such as bacteria and tissue cells.

TESTEE—one, of whom a test is being performed.

What is claimed is:

1. The assemblage of a general and non-specific carcinoma/cancer test kit for at-home use by laypersons and professional individuals alike.

2. A general and non-specific cancer test kit for non-prescribed “over the counter” sale to laypersons and professional individuals alike.

3. A non-specific qualitative carcinoma assay expressing the presence or absence of the antigen; beta hCG/CGH, with the appropriate positive or negative terms.

4. An at home, in-vitro, assay for carcinoma expressing positive and negative identities in identifiable form, to be defined within the illustrated instructions provided with each kit.
A positive result is one that confirms or affirms the presence of the carcinoma biomarker in the test-takers urine. This result would indicate the presence of cancer.
A negative result is one that confirms or affirms the absence of the carcinoma biomarker in the test-takers urine. Testee does not have cancer.

5. An At-Home Cancer Test kit requiring nothing more than that which is provided in the kit purchased, coupled with an un-contaminated sample of the test takers urine.
